(19) United States
(12) Patent Application Publication
(10) Pub. No.: US 2025/0345196 A1
(43) Pub. Date: Nov. 13, 2025

(54) DELIVERY SYSTEM WITH TELESCOPING INNER LUMEN

(71) Applicant: KONINKLIJKE PHILIPS N.V., EINDHOVEN (NL)

(72) Inventor: Scott Robert WILSON, MAPLE GROVE, MN (US)

(21) Appl. No.: 18/871,186

(22) PCT Filed: May 29, 2023
(86) PCT No.: PCT/EP2023/064310

§ 371 Date: Dec. 3, 2024

Related U.S. Application Data
(60) Provisional application No. 63/350,448, filed on Jun. 9, 2022.

(51) Int. Cl. A61F 2/966; A61F 2/95

(52) U.S. Cl. CPC A61F 2/966; A61F 2/9517

(57) ABSTRACT

A delivery device (10) for delivering an associated self-expanding intravascular therapy device (2) into a blood vessel includes an inner sheath (12): an outer sheath (14) having a sheath opening (16) disposed at an end thereof, the inner sheath being disposed coaxially inside the outer sheath and movable through the outer sheath: a handle (24) wherein a proximate end of the outer sheath is disposed inside the handle: a fixed support tube (26) disposed inside the handle and secured to the handle, the fixed support tube being disposed coaxially inside the inner sheath and the inner sheath being movable respective to the fixed support tube; and a gearing mechanism (28) configured to control forward movement of the inner sheath through the outer sheath and backward movement of the outer sheath in an opposing direction from the forward movement of the inner sheath.

### FIELD

The following relates generally to the catheter arts, vascular therapy, lesion treatment arts, and related arts.

### BACKGROUND

Expandable, metal support structures such as stents delivered via intravascular devices are commonly used in the treatment in intravascular disease, arterial and venous, as well as in larger regions of the anatomy such as the esophagus.

Delivery systems used to deploy metal structures typically include catheter components that utilize relative axial motion to deploy the stent. In one design, an outer sheath that constrains the expandable structure is axially retracted (moved towards the user), while in inner support catheter remains static, or is moved forward, in the opposite direction of the outer sheath. In the case of the latter “dual motion deployment,” the forward movement of the inner support catheter compensates for the typical shortening of the length of a stent as it expands when deployed out from the outer sheath. This compensation permits more accurate placement of the stent in the blood vessel. For some current delivery system handle mechanisms, the inner support lumen component includes a luer fitting, and extends proximally out of the handle. During dual motion deployment, this inner lumen/luer moves inward towards the handle, and can become kinked, or entangled with nearby items.

The following discloses certain improvements to overcome these problems and others.

### SUMMARY

In some embodiments disclosed herein, a delivery device for delivering an associated self-expanding intravascular therapy device into a blood vessel includes an inner sheath; an outer sheath having a sheath opening disposed at an end thereof, the inner sheath being disposed coaxially inside the outer sheath and movable through the outer sheath; a handle wherein a proximate end of the outer sheath is disposed inside the handle; a fixed support tube disposed inside the handle and secured to the handle, the fixed support tube being disposed coaxially inside the inner sheath and the inner sheath being movable respective to the fixed support tube; and a gearing mechanism configured to control forward movement of the inner sheath through the outer sheath and backward movement of the outer sheath in an opposing direction from the forward movement of the inner sheath.

In some embodiments disclosed herein, a delivery device for delivering an associated self-expanding intravascular therapy device into a blood vessel includes an inner sheath; an outer sheath having a sheath opening disposed at an end thereof, the inner sheath being disposed coaxially inside the outer sheath and movable through the outer sheath; a handle wherein a proximate end of the outer sheath is disposed inside the handle; a fixed support tube disposed inside the handle and secured to the handle, the fixed support tube being disposed coaxially inside the inner sheath and the inner sheath being movable respective to the fixed support tube; and a gearing mechanism configured to control forward movement of the inner sheath through the outer sheath and backward movement of the outer sheath in an opposing direction from the forward movement of the inner sheath. The fixed support tube has a central lumen of diameter d₁; and the inner sheath has a central lumen of the diameter d₁.

In some embodiments disclosed herein, a delivery device for delivering an associated self-expanding intravascular therapy device into a blood vessel includes an inner sheath; an outer sheath having a sheath opening disposed at an end thereof, the inner sheath being disposed coaxially inside the outer sheath and movable through the outer sheath; a handle wherein a proximate end of the outer sheath is disposed inside the handle; a fixed support tube disposed inside the handle and secured to the handle, the fixed support tube being disposed coaxially inside the inner sheath and the inner sheath being movable respective to the fixed support tube; a gearing mechanism configured to control forward movement of the inner sheath through the outer sheath and backward movement of the outer sheath in an opposing direction from the forward movement of the inner sheath; a luer fitting secured to an end of the fixed support tube extending outside of the handle; a first shuttle configured to prevent movement of the inner sheath out of the outer sheath; and a second shuttle configured to prevent movement of the luer out of the outer sheath.

One advantage resides in providing a catheter delivery device with a telescoping inner lumen that does not variably extend in length out of a catheter handle.

Another advantage resides in providing a catheter delivery device with an outer sheath and an inner sheath that move in opposing directions, without a concomitant variation in the total length of the handle.

Another advantage resides in providing a catheter delivery device with an inner sheath that does not kink during movement.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIGS. 1 and 2 diagrammatically illustrate a sectional view of a vascular therapy device in accordance with the present disclosure, with different positions of a movable inner sheath.

FIGS. 3 and 4 diagrammatically illustrate isolation sectional views of the movable inner sheath (FIG. 3) and the fixed support tube (FIG. 4) of the device of FIG. 1.

FIG. 5 diagrammatically illustrates a method of performing a vascular therapy method using the device of FIG. 1.

### DETAILED DESCRIPTION

A dual motion handle assembly is used in an endovascular procedure in delivering a stent or other device that undergoes foreshortening. For example, if the device is a self-expanding nitinol stent (or a braided stent, or a woven stent), then as it expands upon deployment in the radial direction it will shrink in the longitudinal direction. This longitudinal shrinkage (i.e. “foreshortening”) can lead to misplacement of the stent. The dual motion handle assembly resolves this problem by concurrently drawing back the outer sheath while extending the inner sheath (the “dual motion”) so that the stent position is maintained during the deployment in spite of the foreshortening.

However, as the inner sheath is drawn back, the end with the luer fitting for connecting a tube to the inner sheath extends further and further out of the back end of the handle, and can potentially interfere with handle manipulation.

To address this problem, the following discloses a dual tubular support arrangement. An inner support #1 moves normally during the dual motion deployment operation. A second inner support #2 is coaxially located inside the inner support and is secured to the handle. The luer fitting is connected to inner support #2, and hence has a fixed position along with the fixed inner support #2. The purpose of inner support #2 is to provide a sealed fluid flow path from the inner lumen to the luer fitting regardless of the position of movable inner support #1.

In another aspect, to ensure a (mostly) constant inside diameter (ID) for the fluid path from the inner sheath to the luer fitting, inner support #1 suitably has a smaller ID proximal to the inner sheath which widens to a larger ID that accommodates inner support #2. The smaller ID proximate to the inner sheath is the same as the ID of inner support #2. This provides a constant ID for the fluid path except at the gap between the ID step and the end of inner support #2 when inner support #1 is moved away from inner support #2 as the inner sheath is extended. This larger ID region cannot be avoided, but the ID step of inner support #1 and the end of inner support #2 can be chamfered to provide a sloped transition. This design facilitates insertion of a guide wire or other instrument through the inner sheath without hanging up along the path between the luer fitting and the inner sheath.

While designed for the dual motion catheter, the disclosed design could be used in any instrument having an inner sheath that moves respective to an outer sheath.

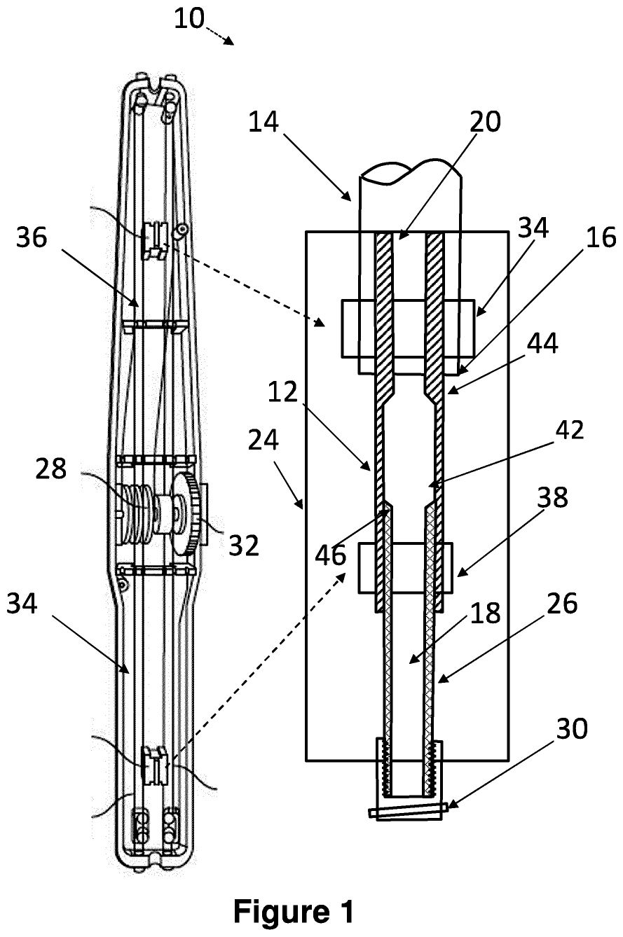

With reference to FIG. 1, an illustrative vascular therapy (i.e., thrombectomy or atherectomy) apparatus 1 is diagrammatically shown. As shown in FIG. 1, the apparatus 1 includes a delivery device 10 for delivering a self-expanding vascular therapy device (e.g., a self-expanding stent, a self-expanding filter, and so forth) into a blood vessel. The delivery device 10 includes an inner sheath 12 surrounded by an outer sheath 14. The inner sheath 12 is disposed coaxially inside the outer sheath 14 and is movable through the outer sheath 14. The inner sheath 12 is movable in a longitudinal forward (i.e., advancement) translation direction and a longitudinal backward (i.e., withdrawal) translation direction within the outer sheath 14. At one end of the outer sheath 14 is a sheath opening 16 through which the distal end of the inner sheath 12 can move into and out of the outer sheath 14.

As shown FIG. 1, the delivery device 10 also includes a handle 24, and a proximate end of the outer sheath 14 is disposed inside the handle 24. A fixed support tube 26 is disposed inside the handle 24 and secured to the handle 24. The fixed support tube 26 is disposed coaxially inside the inner sheath 12, and the inner sheath 12 is movable respective to the fixed support tube 26.

FIG. 1 also shows a gearing mechanism 28 configured to control forward movement of the inner sheath 12 through the outer sheath 14 and backward movement of the outer sheath 14 in an opposing direction from the forward movement of the inner sheath 12. The movement of both the catheter 12 and the containment sheath 14 is relative to the handle 24 (i.e., into, out of, or through the handle 24). The gearing mechanism 28 can be disposed on or in a portion of the handle 24.

A luer fitting 30 is secured to an end of the fixed support tube 26 extending outside of the handle 24. The fixed support tube 26 is disposed coaxially inside the inner sheath 12, and forms a sealed fluid flow path from the inner sheath to the luer fitting 30.

FIG. 1 also shows the delivery device 10 with a portion of the handle 24 (i.e., a cover) removed, and internal components of the delivery device 10 are shown. The gearing mechanism 28 is configured to adjust a ratio of a speed of the forward movement of the inner sheath 12 and a speed of the backward movement of the outer sheath 14. To do so, the gearing mechanism 28 can include a thumbwheel 32 disposed externally on a portion of the handle 24. The thumbwheel 32 is operatively connected or engaged with one or more components disposed in internally in a portion of the handle 24, for example by way of control wires, to drive movement of the inner sheath 12 and the outer sheath 14. As shown in FIG. 1, the gearing mechanism 28 includes a first shuttle 34 connected to the inner sheath 12 and the outer sheath 14, and configured to drive backward movement of the outer sheath 14 relative to the handle 24 while a user manually moves the thumbwheel 32 by way of a first control wire 36. The first shuttle 34 is connected to the outer sheath 14, and is configured to pull the outer sheath 14 back relative to the handle 24 while a user moves the thumbwheel 32 by way of first control wire 36. A second shuttle 38 is connected to the inner sheath 12, and is configured to drive forward movement of the inner sheath 12 relative to the handle 24 while a user manually moves the thumbwheel 32 by way of a second control wire 40 . . . . The fixed support tube 26 is fixed to the handle 24 to prevent movement thereof.

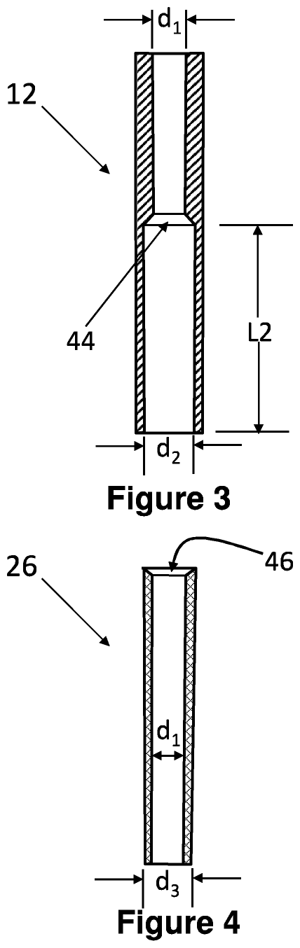

As shown in FIG. 1, the fixed support tube 26 has a central lumen 18 of diameter d₁, and the inner sheath 12 has a central lumen 20 of the diameter d₁ (i.e., the same diameter as the central lumen 18). In one example, the diameter d₁ of the central lumen 20 of the inner sheath 12 is sized to receive a self-expanding vascular therapy device (not shown) at a distal end thereof that is inserted into the patient in an intravascular therapy procedure. In another example, an outer diameter (not shown) of the inner sheath 12 is sized to fit the vascular therapy device (where a distal portion of the inner sheath 12 is stepped) to allow the vascular therapy device to be disposed between the inner sheath 12 and the outer sheath 12. The step acts as a stop to push against the vascular therapy device while the outer sheath 14 is retracted. A portion of the inner sheath 12 proximate to the fixed support tube 26 has an increased diameter d₂ of its central lumen where d₂ is larger than d₁ (depicted with reference character 42; see also FIG. 3). The fixed support tube 26 has an outer diameter d₂ that is equal to the diameter d₂ of the portion of the central lumen 20 of the inner sheath 12 proximate to the fixed support tube 26. A transition (labeled in FIG. 3) of the central lumen 20 of the inner sheath 12 from the diameter d₁ to the diameter d₂ comprises a sloped chamfer (depicted with reference character 44). The end of the fixed support tube 26 disposed coaxially inside the inner sheath 12 has a sloped chamfer (depicted with reference character 46).

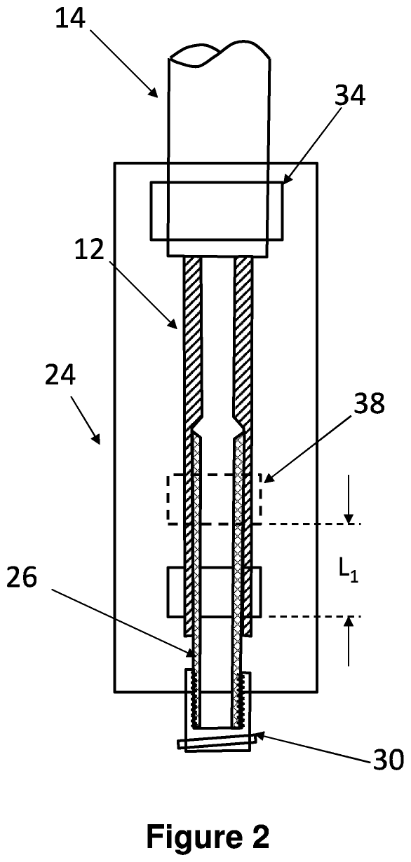

With continuing reference to FIG. 1, FIG. 2 shows an example movement of the inner sheath 12 and the outer sheath 14 relative to each other. As described above, the outer sheath 14 is moved back, while the inner sheath 12 is moved forward. In order to accomplish this movement, the inner sheath 12 must extend outside the handle 24 by a length “L1” to compensate for the forward movement. In the absence of the fixed support tube 26, the luer fitting would typically connect directly to the movable inner sheath, and the luer fitting would then move inward or outward from the end of the handle by an amount equal to the extended length of the movable inner sheath. This variably extending luer fitting can become caught on other items, pinched, or kinked during use.

However, as seen by comparing FIGS. 1 and 2, in the design of FIGS. 1 and 2 the movement of the movable inner sheath 12 during deployment of the stent or other therapy device does not extend outside of the handle 24. This is because the luer fitting 30 is secured to the fixed support tube 26 which is fixed to thee handle 24. The fixed support tube 26 forms a fluid-tight seal with the movable inner sheath 12. The movement of the proximal end of the inner sheath 12 is thus contained wholly within the handle 24. Aspirated blood or other fluid flowing through the central lumen of the movable inner sheath 12 flows on through the fixed support tube 26 to the luer fitting 30, so that the luer fitting 30 remains in fixed position during movement of the movable inner sheath 12 during the deployment process, and yet also remains in fluid communication with the central lumen of the movable inner sheath 12.

As shown in FIG. 2, the fixed support tube 26 is fixed to the handle 24, and is slidably inserted into the inner sheath 12. When activating the thumbwheel 32, the second shuttle 38 moves forward, which then moves the inner sheath 12, while the fixed support tube 26 remains static. This allows the dual motion delivery, without the fixed support tube 26 moving to extend out of the handle 24. In some examples, the first shuttle 34 can move proximally towards the fixed support tube 26.

FIG. 3 shows a sectional view of the inner sheath 12 in isolation, with the diameters d₁ and d₂ labeled along with the sloped chamfer 44. The inner sheath 12 is a primary inner support lumen for the delivery device 10, running the entire length of the catheter from the handle (shown in FIGS. 1 and 2) to the distal end of the catheter (not shown) containing the implantable expanding stent, to the handle 24. The inside diameter of the inner sheath 12 is stepped at the proximal end to form sloped chamfer 44 to allow insertion of fixed support tube 26 into the portion of the movable inner sheath 12 with diameter d₂. The length L2 of the portion of the inner sheath 12 with the widened diameter d₂ is dependent on (and should be equal to or greater than) the maximum length L1 of the movement of the movable inner sheath 12 required to deploy the stent.

The inner sheath 12 can be manufactured as a single component, with a step (i.e., the sloped chamfer 44) by drilling or machining. Alternatively, the inner sheath 12 can be made from multiple single lumen tube components bonded to together to create the sloped chamfer 44. At the location of the sloped chamfer 44, the stepped surface can be chamfered to facilitate insertion of auxiliary devices (guidewires) during the procedure.

FIG. 4 shows a sectional view of the fixed support tube 26 in isolation, with the sloped chamfered end 46 labeled. The fixed support tube 26 includes a single lumen tube, sufficiently long enough to slide into the inner sheath 12 over the distance of L1 (L2≥L1), and with the following diameter requirements: an inner diameter d₁ equal to the inner diameter d₁ of the inner support 12, and an outer diameter da that is equal to the inner diameter of the step section of Inner Support 1 (d₂). In a variant embodiment, to provide a slightly compressive fitting to improve fluid seal between the fixed support tube 26 of outer diameter d₃ and the widened inner lumen of diameter d₂ of the widened portion of the movable inner sheath 12, it is contemplated for the outer diameter da of the fixed support tube 26 to be slightly larger than the lumen diameter d₂ of the widened lumen of the movable sheath 12. In such a design variant, the fixed support tube 26 is slightly compressed inside the widened lumen of the movable sheath 12. However, the advantage of such compression in improving the fluid seal is to be balanced against the friction thereby introduced which can interfere with the movement of the inner sheath 12 during deployment of the therapy device. The optimal balance depends on factors such as the coefficient of friction between the materials of the two tubes 12 and 26 and the compressibility of those materials. Materials for the inner sheath 12 and the fixed support tube 26 should be lubricous (low friction coefficient) to allow a close tolerance to fit, yet still allow the components to easily slide against each other (e.g. PEEK, high durometer PEBAX with lubricious filler, PTFE). If a tight seal is required to prevent leakage, an O-ring seal could also be inserted between the components, in which case outer diameter da could be slightly less than lumen diameter d₂ with the O-ring bridging the gap.

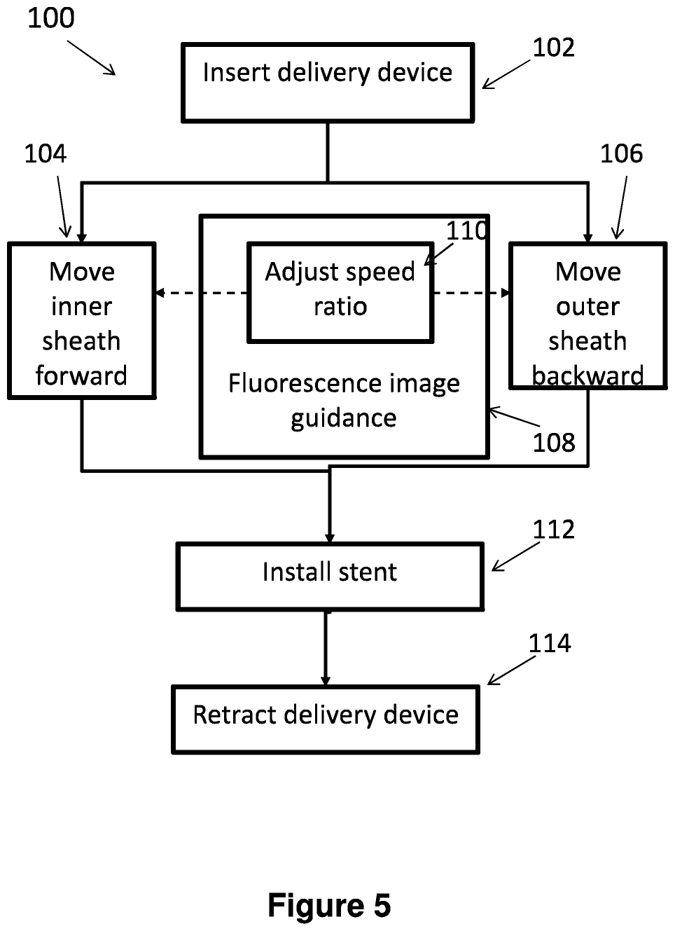

FIG. 5 shows an example of a flowchart showing a vascular therapy method 100 using the therapy apparatus 1. To begin the method 100, in an operation 102 the delivery device 10 is inserted into a target tissue (e.g., an artery or vein or esophagus of the patient). When the device 10 is at the delivery site, at an operation 104, the inner sheath 12 is moved forward in the target tissue. At an operation 106 performed simultaneously with the operation 104, the outer sheath 14 is moved backwards in the target tissue. In an operation 108 concurrent with the simultaneous operations 104, 106, the operator can observe the deployment of the self-expanding vascular therapy device via fluorescence image guidance or another suitable imaging modality. The fluorescence image-guidance 108 preferably images the self-expanding vascular therapy device 2 with sufficient resolution of to enable the operator to see the extent of (or lack of) bunching of the self-expanding vascular therapy device 2 during the delivery. At an operation 110, the operator adjusts the ratio of the speed of the forward movement of the catheter in operation 104 and the speed of the backward movement of the containment sheath in the operation 106 to achieve a desired amount of bunching, with more bunching being provided in areas where the operator decides the self-expanding vascular therapy device 2 should have greater stiffness and less (or no) bunching in areas where the operator decides the self-expanding vascular therapy device 2 should have less stiffness. The concurrent operations 104, 106, 108, 110 are performed until the self-expanding vascular therapy device 2 is completely deployed at an operation 112, after which in an operation 114 the delivery device 10 is retracted (e.g., withdrawn from the artery into which it was inserted).

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A delivery device for delivering an associated self-expanding intravascular therapy device into a blood vessel, the delivery device comprising:
an inner sheath;
an outer sheath having a sheath opening disposed at an end thereof, the inner sheath being disposed coaxially inside the outer sheath and movable through the outer sheath;
a handle wherein a proximate end of the outer sheath is disposed inside the handle;
a fixed support tube disposed inside the handle and secured to the handle, the fixed support tube being disposed coaxially inside the inner sheath and the inner sheath being movable respective to the fixed support tube; and
a gearing mechanism configured to control forward movement of the inner sheath through the outer sheath and backward movement of the outer sheath in an opposing direction from the forward movement of the inner sheath.

2. The delivery device of claim 1, further comprising:
a luer fitting secured to an end of the fixed support tube extending outside of the handle.

3. The delivery device of claim 2, wherein the fixed support tube is disposed coaxially inside the inner sheath forms a sealed fluid flow path from the inner sheath to the luer fitting.

4. The delivery device of claim 1, wherein:
the gearing mechanism is disposed on or in a portion of the handle.

5. The delivery device of claim 1, wherein:
the fixed support tube has a central lumen of diameter d₁; and
the inner sheath has a central lumen of the diameter d₁.

6. The delivery device of claim 5, wherein a portion of the inner sheath proximate to the fixed support tube has an increased diameter d₂ of its central lumen where d₂ is larger than d₁.

7. The delivery device of claim 6, wherein the fixed support tube has an outer diameter d₂ that is equal to the diameter d₂ of the portion of the central lumen of the inner sheath proximate to the fixed support tube.

8. The delivery device of claim 7, wherein a transition of the central lumen of the inner sheath from the diameter d₁ to the diameter d₂ comprises a sloped chamfer.

9. The delivery device of claim 8, wherein the end of the fixed support tube disposed coaxially inside the inner sheath has a sloped chamfer.

10. The delivery device of claim 4, wherein the inner sheath and the outer sheath are sized to receive a self-expanding vascular therapy device therebetween.

11. The delivery device of claim 1, further comprising:
a first shuttle configured to prevent movement of the inner sheath out of the outer sheath; and
a second shuttle configured to prevent movement of the luer out of the outer sheath.

12. A delivery device for delivering an associated self-expanding intravascular therapy device into a blood vessel, the delivery device comprising:
an inner sheath;
an outer sheath having a sheath opening disposed at an end thereof, the inner sheath being disposed coaxially inside the outer sheath and movable through the outer sheath;
a handle wherein a proximate end of the outer sheath is disposed inside the handle;
a fixed support tube disposed inside the handle and secured to the handle, the fixed support tube being disposed coaxially inside the inner sheath and the inner sheath being movable respective to the fixed support tube; and
a gearing mechanism configured to control forward movement of the inner sheath through the outer sheath and backward movement of the outer sheath in an opposing direction from the forward movement of the inner sheath;
wherein:
the fixed support tube has a central lumen of diameter d₁; and
the inner sheath has a central lumen of the diameter d₁.

13. The delivery device of claim 12, further comprising:
a luer fitting secured to an end of the fixed support tube extending outside of the handle.

14. The delivery device of claim 13, wherein the fixed support tube is disposed coaxially inside the inner sheath forms a sealed fluid flow path from the inner sheath to the luer fitting.

15. The delivery device of claim 12, wherein:
the gearing mechanism is disposed on or in a portion of the handle.

16. The delivery device of claim 12, wherein a portion of the inner sheath proximate to the fixed support tube has an increased diameter d₂ of its central lumen where d₂ is larger than d₁.

17. The delivery device of claim 16, wherein the fixed support tube has an outer diameter d₂ that is equal to the diameter d₂ of the portion of the central lumen of the inner sheath proximate to the fixed support tube.

18. The delivery device of claim 17, wherein a transition of the central lumen of the inner sheath from the diameter d₁ to the diameter d₂ comprises a sloped chamfer.

19. The delivery device of claim 18, wherein the end of the fixed support tube disposed coaxially inside the inner sheath has a sloped chamfer.

20. A delivery device for delivering an associated self-expanding intravascular therapy device into a blood vessel, the delivery device comprising:
an inner sheath;
an outer sheath having a sheath opening disposed at an end thereof, the inner sheath being disposed coaxially inside the outer sheath and movable through the outer sheath;
a handle wherein a proximate end of the outer sheath is disposed inside the handle;
a fixed support tube disposed inside the handle and secured to the handle, the fixed support tube being disposed coaxially inside the inner sheath and the inner sheath being movable respective to the fixed support tube;
a gearing mechanism configured to control forward movement of the inner sheath through the outer sheath and backward movement of the outer sheath in an opposing direction from the forward movement of the inner sheath;
a luer fitting secured to an end of the fixed support tube extending outside of the handle;
a first shuttle configured to prevent movement of the inner sheath out of the outer sheath; and
a second shuttle configured to prevent movement of the luer out of the outer sheath.
